(19) United States
(12) Patent Application Publication
(10) Pub. No.: US 2013/0190346 A1
(43) Pub. Date: Jul. 25, 2013

(54) METHOD OF REDUCING CNS AND GASTROINTESTINAL SIDE AFFECTS ASSOCIATED WITH LONG-TERM, DEXTROMETHORPHAN/LOW-DOSE QUINIDINE COMBINATION THERAPY

(71) Applicant: Avanir Pharmaceuticals, Inc., Aliso Viejo, CA (US)

(72) Inventors: Gregory J. Flesher, Aliso Viejo, CA (US); Randall E. Kaye, Aliso Viejo, CA (US)

(73) Assignee: Avanir Pharmaceuticals, Inc., Aliso Viejo, CA (US)

(21) Appl. No.: 13/752,958

(22) Filed: Jan. 29, 2013

Related U.S. Application Data
(63) Continuation of application No. 12/853,572, filed on Aug. 10, 2010, now abandoned.
(60) Provisional application No. 61/238,045, filed on Aug. 28, 2009.

(51) Int. Cl. A61K 31/485; A61K 31/439

(52) U.S. Cl. CPC A61K 31/485; A61K 31/439
USPC 514/289

(57) ABSTRACT

Pharmaceutical compositions and methods for treating neurological disorders by administering same are provided. The compositions comprise dextromethorphan in combination with quinidine. This invention also provides methods of reducing CNS and gastrointestinal side effects associated with a long term, dextromethorphan/low-dose quinidine combination therapy.

### CROSS-REFERENCES TO RELATED APPLICATIONS

This application is a continuation of U.S. application Ser. No. 12/853,572, filed Aug. 10, 2010, which claims the benefit under 35 U.S.C. §1.119(e) of U.S. provisional Application No. 61/238,045, filed Aug. 28, 2009, the contents of which are incorporated by reference in their entirety.

### BACKGROUND OF THE INVENTION

This invention relates to improvements upon a previously known drug combination therapy comprising dextromethorphan as the active ingredient with quinidine to enhance the half-life of dextromethorphan. The improvements reduce adverse side effects. The combination therapy comprising dextromethorphan and quinidine have been used for treatment of emotional lability and pseudobulbar affect (US5206248 to Smith), and chronic or intractable pain, tinnitus and sexual dysfunction (US5863927 to Smith). The combination therapy methods disclosed in these patents use dextromethorphan with a high-dose quinidine formulation, e.g., a daily dose of quinidine from about 50 mg to about 300 mg. A low-dose quinidine formulation of dextromethorphan/quinidine combination therapy has also been used for treatment of emotional lability or pseudobulbar affect (U.S. application Ser. No. 11/035,213). The low-dose quinidine claims require a daily dose of dextromethorphan from about 20 to 80 mg and of quinidine from about 10-30 mg wherein the ratio of dextromethorphan to quinidine cannot exceed a w/w ratio of 1:0.5.

Patients suffering from neurodegenerative diseases or brain damage such as is caused by stroke or head injury often are afflicted with emotional problems associated with the disease or injury. The terms emotional lability and pseudobulbar affect are used by psychiatrists and neurologists to refer to a set of symptoms that are often observed in patients who have suffered a brain insult such as a head injury, stroke, brain tumor, or encephalitis, or who are suffering from a progressive neurodegenerative disease such as Amyotrophic Lateral Sclerosis (ALS, also called motor neuron disease or Lou Gehrig's disease), Parkinson's disease, Alzheimer's disease, or multiple sclerosis. In the great majority of such cases, emotional lability occurs in patients who have bilateral damage (damage which affects both hemispheres of the brain) involving subcortical forebrain structures.

Emotional lability, which is distinct from clinical forms of reactive or endogenous depression, is characterized by intermittent spasmodic outbursts of emotion (usually manifested as intense or even explosive crying or laughing) at inappropriate times or in the absence of any particular provocation. Emotional lability or pseudobulbar affect is also referred to by the terms emotionalism, emotional incontinence, emotional discontrol, excessive emotionalism, and pathological laughing and crying. The feelings that accompany emotional lability are often described in words such as “disconnectedness,” since patients are fully aware that an outburst is not appropriate in a particular situation, but they do not have control over their emotional displays.

Emotional lability or pseudobulbar affect becomes a clinical problem when the inability to control emotional outbursts interferes in a substantial way with the ability to engage in family, personal, or business affairs. For example, a businessman suffering from early-stage ALS or Parkinson's disease might become unable to sit through business meetings, or a patient might become unable to go out in public, such as to a restaurant or movie, due to transient but intense inability to keep from crying or laughing at inappropriate times in front of other people. These symptoms can occur even though the patient still has more than enough energy and stamina to do the physical tasks necessary to interact with other people. Such outbursts, along with the feelings of annoyance, inadequacy, and confusion that they usually generate and the visible effects they have on other people, can severely aggravate the other symptoms of the disease; they lead to feelings of ostracism, alienation, and isolation, and they can render it very difficult for friends and family members to provide tolerant and caring emotional support for the patient.

Patients in need of dextromethorphan/quinidine combination therapy include patients suffering from emotional lability and other chronic disorders, such as chronic pain. Dextromethorphan/quinidine combination therapy provides at least some degree of improvement compared to other known drugs, in at least some patients. Patients in need of dextromethorphan/quinidine combination therapy also include patients suffering from neurologic impairment, such as a progressive neurologic disease.

Dextromethorphan typically has dose-dependent CNS and gastrointestinal side effects. Quinidine is also known to be associated with a number of side effects. During the course of this invention, it is surprisingly and unexpectedly discovered that a sub-optimal combination dose of for a period of no less than 7 days and no more than 20 days prior to increasing the dose of dextromethorphan to a therapeutically beneficial amount would result in dramatic reduction of these side effects.

### BRIEF SUMMARY OF THE INVENTION

The present invention is further directed to a method of reducing Central Nervous System (CNS) and gastrointestinal side effects associated with a long term, dextromethorphan/low-dose quinidine combination therapy by permitting a patient to acclimate to dextromethorphan, the method comprising administration of a sub-optimal combination dose for a period of no less than 7 days and no more than 20 days prior to increasing the dose of dextromethorphan to a therapeutically beneficial amount, wherein the sub-optimal combination dose comprises dextromethorphan from about 10 mg/day to about 30 mg/day and quinidine from about 5 mg/day to less than about 15 mg/day with the proviso that the weight to weight ratio of dextromethorphan to quinidine is 1:0.75 or less of quinidine. In other words, for each weight unit of dextromethorphan, there should be no more than ¾ unit of quinidine. In some embodiments, the weight to weight ratio of dextromethorphan to quinidine is 1:0.5 or less of quinidine. In other words, for each weight unit of dextromethorphan, there should be no more than ½ unit of quinidine.

In some embodiments, the method of reducing CNS and gastrointestinal side effects associated with a long term, dextromethorphan/low-dose quinidine combination therapy is used to reduce nausea. In some embodiments, the method of reducing CNS and gastrointestinal side effects associated with a long term, dextromethorphan/low-dose quinidine combination therapy is used to reduce dizziness. In some embodiments, the method of reducing CNS and gastrointestinal side effects associated with a long term, dextromethorphan/low-dose quinidine combination therapy is used to reduce fatigue.

In some embodiments, the sub-optimal combination dose is administered as one combined dose per day. In some embodiments, the sub-optimal combination dose is administered as at least two combined dose per day. In some embodiments, the sub-optimal combination dose comprises the dextromethorphan and the quinidine administered in separate doses.

In some embodiments, the sub-optimal combination dose is administered for a period of 7 days. In some embodiments, the sub-optimal combination dose is administered for a period of 14 days.

In some embodiments, the sub-optimal combination dose comprises dextromethorphan from about 10 mg/day to about 20 mg/day. In some embodiments, the sub-optimal combination dose comprises dextromethorphan from about 20 mg/day to about 30 mg/day. In some embodiments, the sub-optimal combination dose comprises dextromethorphan about 10 mg/day. In some embodiments, the sub-optimal combination dose comprises dextromethorphan about 20 mg/day. In some embodiments, the sub-optimal combination dose comprises dextromethorphan about 30 mg/day. In some embodiments, the sub-optimal combination dose comprises quinidine from about 5 mg/day to about 10 mg/day. In some embodiments, the sub-optimal combination dose comprises quinidine from about 10 mg/day to about 15 mg/day. In some embodiments, the sub-optimal combination dose comprises quinidine about 5 mg/day. In some embodiments, the sub-optimal combination dose comprises quinidine about 10 mg/day. In some embodiments, the sub-optimal combination dose comprises quinidine about 15 mg/day. In some embodiments, the sub-optimal combination dose comprises dextromethorphan about 30 mg/day and quinidine about 10 mg/day. In some embodiments, the sub-optimal combination dose comprises dextromethorphan about 20 mg/day and quinidine about 10 mg/day.

In some embodiments, the method of the present invention reduces CNS and gastrointestinal side effects associated with a long term, dextromethorphan/low-dose quinidine combination therapy in treatment of emotional lability or pseudobulbar effect. In some embodiments, the emotional lability or pseudobulbar effect is caused by a neurodegenerative disease or condition or a brain injury.

In some embodiments, the sub-optimal combination dose is one third of the therapeutically beneficial amount. In some embodiments, the sub-optimal combination dose is 50% of the therapeutically beneficial amount.

In another aspect, the present invention provides a kit for reducing Central Nervous System (CNS) and gastrointestinal side effects associated with a long term, dextromethorphan/low-dose quinidine combination therapy, comprising: (a) a sub-optimal combination dose for a period of no less than 7 days and no more than 20 days comprising dextromethorphan from about 10 mg/day to about 30 mg/day and quinidine from about 5 mg/day to about 15 mg/day with the proviso that the weight to weight ratio of dextromethorphan to quinidine is 1:0.75 or less of quinidine; and (b) a therapeutically beneficial dose for a period of 7 days or more. In some embodiments, the weight to weight ratio of dextromethorphan to quinidine is 1:0.5 or less of quinidine.

In some embodiments, the sub-optimal combination dose of the kit is one third of the therapeutically beneficial dose. In some embodiments, the sub-optimal combination dose of the kit is 50% of the therapeutically beneficial dose.

### DEFINITIONS

As used herein, the term “side effect” refers to an undesired consequence other than the one(s) for which an agent or measure is used, as the adverse effects produced by a drug, especially on a tissue or organ system other then the one sought to be benefited by its administration.

As used herein, the term “CNS side effects” or “central nerve system side effects” refers to side effects associated with central nerve system. Exemplary CNS side effects include, but are not limited to, nervousness, dizziness, sleeplessness, light-headedness, tremor, hallucinations, convulsions, CNS depression, fear, anxiety, headache, increased irritability or excitement, tinnitus, drowsiness, dizziness, sedation, somnolence, confusion, disorientation, lassitude, incoordination, fatigue, euphoria, nervousness, insomnia, convulsive seizures, excitation, catatonic-like states, hysteria, hallucinations, and extrapyramidal symptoms such as oculogyric crisis, torticollis, hyperexcitability, increased muscle tone, ataxia, and tongue protrusion.

As used herein, the term “GI side effects” or “gastrointestinal side effects” refers to side effects associated with gastrointestinal system. Exemplary gastrointestinal side effects include, but are not limited to, nausea, vomiting, abdominal pain, dysphagia, dyspepsia, diarrhea, abdominal distension, flatulence, peptic ulcers with bleeding, loose stools, constipation, stomach pain, heartburn, gas, loss of appetite, feeling of fullness in stomach, indigestion, bloating, hyperacidity, dry mouth, gastrointestinal disturbances, and gastric pain.

Side effects can be assessed by the methodologies known in the art. For example, nausea can be measured using a discrete scale (DS), a visual analogue scale (VAS) and a continuous chromatic analogue scale (ACCS), and evaluated according to 4 different dimensions such as maximal intensity, entity, duration and quantity (Favero et al., Assessment of nausea, European Journal of Clinical Pharmacology, 38:115-120, 2004.). Nausea can be measured across individuals and situations by measuring multiple dimensions of nausea (Muth et al., Assessment of the multiple dimensions of nausea: the Nausea Profile, Journal of Psychosomatic Research, 40:511-520, 1996). Single or multiple dimensional approaches to assessment of fatigue have been adopted and used extensively in the field, including physical, cognitive, emotional and functional assessment (Hjollund et al., Assessment of fatigue in chronic disease: a bibliographic study of fatigue measurement scales, Health and quality of life outcome, 5:12, 2007). Characterization of generic and disease-specific fatigue have been developed and applied in the field (see, Munch et al., Multidimensional measurement of fatigue in advanced cancer patients in palliative care: an application of the multidimensional fatigue inventory, Journal of Pain and symptom management, 31:533-541, 2006; Measurement of fatigue in Systemic Lupus Erythematosus: a systematic review, 57:1348-1357, 2007; Bowman, et al., Measurement of fatigue and discomfort in primary Sjogren's syndrome using a new questionnaire tool, 43:758-764, 2004.). Similarly, dizziness can also be assessed and evaluated according to various methods. For example, an assay for the assessment of drug side effects, particularly the side effect of dizziness, has been reported (EP1755452). Werner Institute of Balance and Dizziness has also developed several tests to assess dizziness, e.g., a vestibular auto-rotation test (http:www.nomorevertigo.com/services-testing-performed.html).

As defined herein, the term “long term” refers to a period of dextromethorphan/quinidine combination therapy for at least one month. In some embodiments, a long term combination therapy lasts for at least 2, 3, 4, 5, 6, 7, 8, 9, 10, or 11 months. In some embodiments, a long term combination therapy lasts for at least 1, 2, 3, 4, or 5 years. In some embodiments, a long term combination therapy lasts for longer than 5 years.

As defined herein, the term “low dose dextromethorphan/quinidine combination therapy” refers to dextromethorphan/quinidine combination therapy comprising administering to a patient in need thereof dextromethorphan in combination with quinidine, wherein the amount of dextromethorphan administered comprises from about 20 mg/day to about 80 mg/day and wherein the amount of quinidine administered comprises from about 10 mg/day to less than about 30 mg/day and optionally with the proviso that the weight to weight ratio of dextromethorphan to quinidine is 1:0.75 or less of quinidine. In some embodiments, the weight to weight ratio of dextromethorphan to quinidine is 1:0.5 or less of quinidine. In some embodiments, the amount of quinidine administered comprises from about 20 mg/day to about 30 mg/day. In some embodiments, the amount of dextromethorphan administered comprises from about 20 mg/day to about 60 mg/day. In some embodiments, the quinidine comprises quinidine sulfate and the dextromethorphan comprises dextromethorphan hydrobromide, and wherein an amount of quinidine sulfate administered comprises from about 10 mg/day to 30 mg/day and wherein an amount of dextromethorphan hydrobromide administered comprises from about 30 mg/day to about 90 mg/day. In some embodiments, the dextromethorphan and the quinidine are administered in a combined dose, and wherein a weight ratio of dextromethorphan to quinidine in the combined dose is about 1:1.25 or less. In some embodiments, the amount of quinidine administered is from about 10 mg/day to about 45 mg/day. In some embodiments, the amount of quinidine administered is from about 10 mg/day to about 30 mg/day. In some embodiments, the amount of quinidine administered is from about 10 mg/day to about 20 mg/day. In some embodiments, about 20 mg quinidine sulfate is administered per day. In some embodiments, about 60 mg dextromethorphan hydrobromide is administered per day. In some embodiments, about 30 mg quinidine sulfate is administered per day. In some embodiments, about 60 mg dextromethorphan hydrobromide is administered per day. In some embodiments, about 40 mg dextromethorphan hydrobromide is administered per day. In some embodiments, the weight ratio of dextromethorphan to quinidine is about 1:0.75 or less of quinidine. In some embodiments, the dextromethorphan and quinidine are administered in separate doses. In some embodiments, the dextromethorphan and the quinidine are administered as one combined dose per day. In some embodiments, the dextromethorphan and the quinidine are administered as at least two combined doses per day. In some embodiments, the low dose dextromethorphan/quinidine combination therapy is for treating pseudobulbar affect or emotional lability. In some embodiments, the pseudobulbar affect or emotional lability is caused by a neurodegenerative disease or condition or a brain injury.

As defined herein, the term “sub-optimal dose” or “sub-optimal combination dose” refers to a dose below the recommended dose for the dextromethorphan/quinidine combination therapy. In some embodiments, the term “sub-optimal dose” or “sub-optimal combination dose” refers to a dose below a “low dose” used in the dextromethorphan/quinidine combination therapy as defined herein. In some embodiments, the term “sub-optimal dose” or “sub-optimal combination dose” refers to a dose below a therapeutically beneficial amount as defined herein.

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

### DETAILED DESCRIPTION OF THE INVENTION

This invention discloses the surprising discovery that patients taking dextromethorphan/quinidine combination therapy can have reduced adverse side effects by acclimating to the dextromethorphan via a period of sub-optimal doses for one week to 20 days prior to the administration of a therapeutically beneficial amount of a dextromethorphan/quinidine combination dose.

Emotional lability or pseudobulbar affect is associated with a number of neurological diseases, such as stroke (House et al., BMJ, 1989; 298:991-4), multiple sclerosis (MS) (Cotrell et al., J. Neurol. Psychopathol., 1926; 7:1-30; Feinstein et al., Arch. Neurol., 1997; 54:1116-21), amyotrophic lateral sclerosis (ALS) (Miller et al., Neurol., 1999; 52:1311-23; Jackson et al., Semin. Neurol. 1998; 18:27-39; Poeck, K., Pathophysiology of emotional disorders associated with brain damage. In: P. J. Vinken, G. W. Bruyn, editors. Handbook of Clinical Neurology. Amsterdam: North-Holland Publishing Company 1969; pp. 343-67), Alzheimer's disease (Starkstein et al., J. Neurol. Neurosurg. Psychiatry, 1995; 59:55-64), and traumatic brain injury (Brooks, N., Acta Neurochirurgica Suppl., 44 1988; 59-64). Studies have suggested that pseudobulbar affect occurs in up to 50% of patients with ALS (Gallagher, J. P., Acta Neurol. Scand. 1989; 80:114-7).

Emotional lability or pseudobulbar affect in the context of neurological injury can be considered a disconnection syndrome resulting from loss of cortical communication with the brainstem or cerebellum Wilson SAK, J. Neurol. Psychopathol., 1924; IV:299-333; Parvivzi et al., Brain, 2001; 124:1708-19). At the neurotransmitter level, disruptions of ascending and descending serotonergic pathways arising in the brainstem, and dysregulation of dopaminergic projections to the striatum and cortex have been implicated (Andersen et al., Stroke, 1994; 25:1050-2; Ross et al., J. Nerv. Ment. Dis., 1987; 175:165-72; Shaw et al., Brain Sciences in Psychiatry, London: Butterworth, 1982; Udaka et al., Arch. Neurol. 1984; 41:1095-6).

The chemistry of dextromethorphan and its analogs is described in various references such as Rodd, E. H., Ed., Chemistry of Carbon Compounds, Elsevier Publ., N.Y., 1960; Goodman and Gilman's Pharmacological Basis of Therapeutics; Choi, Brain Res., 1987, 403: 333-336; and U.S. Pat. No. 4,806,543. Its chemical structure is as follows:

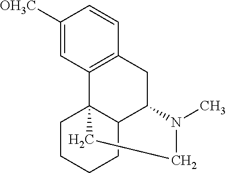

Dextromethorphan is the common name for (+)-3-methoxy-N-methylmorphinan. It is one of a class of molecules that are dextrorotatory analogs of morphine-like opioids. The term “opiate” refers to drugs that are derived from opium, such as morphine and codeine. The term “opioid” is broader. It includes opiates, as well as other drugs, natural or synthetic, which act as analgesics and sedatives in mammals.

Most of the addictive analgesic opiates, such as morphine, codeine, and heroin, are levorotatory stereoisomers (they rotate polarized light in the so-called left-handed direction). They have four molecular rings in a configuration known as a “morphinan” structure, which is depicted as follows:

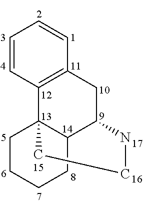 [SMILES: c1ccc2c(c1)C[C@@H]1NCC[C@]23CCCCC13]

In this depiction, the carbon atoms are conventionally numbered as shown, and the wedge-shaped bonds coupled to carbon atoms 9 and 13 indicate that those bonds rise out of the plane of the three other rings in the morphinan structure. Many analogs of this basic structure (including morphine) are pentacyclic compounds that have an additional ring formed by a bridging atom (such as oxygen) between the number 4 and 5 carbon atoms.

An increasing body of evidence indicates dextromethorphan has therapeutic potential for treating several neuronal disorders (Zhang et al., Clin. Pharmacol. Ther. 1992; 51: 647-655; Palmer G C, Curr. Drug Targets, 2001; 2: 241-271; and Liu et al., J. Pharmacol. Exp. Ther. 2003; 21: 21; Kim et al., Life Sci., 2003; 72: 769-783). Pharmacological studies demonstrate that DM is a noncompetitive NMDA antagonist that has neuroprotective, anticonvulsant and antinociceptive activities in a number of experimental models (Desmeules et al., J. Pharmacol. Exp. Ther., 1999; 288: 607-612). In addition to acting as an NMDA antagonist, both DM and its primary metabolite, dextrorphan, bind to sigma-1 sites, inhibit calcium flux channels and interact with high voltage-gated sodium channels (Dickenson et al., Neuropharmacology, 1987; 26: 1235-1238; Carpenter et al., Brain Res., 1988; 439: 372-375; Netzer et al., Eur. J. Pharmacol., 1993; 238: 209-216). Recent reports indicate that an additional neuroprotective mechanism of DM may include interference with the inflammatory responses associated with some neurodegenerative disorders that include Parkinson's disease and Alzheimer's disease (Liu et al., J. Pharmacol. Exp. Ther., 2003; 21: 21). The potential efficacy of DM as a neuroprotectant was explored in limited clinical trials in patients with amyotrophic lateral sclerosis (Gredal et al., Acta Neurol. Scand. 1997; 96: 8-13; Blin et al., Clin. Neuropharmacol., 1996; 19: 189-192) Huntington's disease (Walker et al., Clin. Neuropharmacol., 1989; 12: 322-330) and Parkinson's Disease (Chase et al., J. Neurol., 2000; 247 Suppl 2: 1136-42). DM was also examined in patients with various types of neuropathic pain (Mcquay et al., Pain, 1994; 59: 127-133; Vinik A I, Am. J. Med., 1999; 107: 17S-26S; Weinbroum et al., Can. J. Anaesth., 2000; 47: 585-596; Sang et al., Anesthesiology, 2002; 96: 1053-1061; Heiskanen et al., Pain, 2002; 96: 261-267; Ben Abraham et al., Clin. J. Pain, 2002; 18: 282-285; Sang C N, J. Pain Symptom Manage., 2000; 19: S21-25). Although the pharmacological profile of DM points to clinical efficacy, most clinical trials have been disappointing with equivocal efficacy for DM compared to placebo treatment.

The limited benefit seen with DM in early clinical trials was associated with rapid hepatic metabolism that limits systemic drug concentrations. In one trial in patients with Huntington's disease, plasma concentrations were undetectable in some patients after DM doses that were eight times the maximum antitussive dose (Walker et al., Clin. Neuropharmacol., 1989; 12: 322-330).

DM undergoes extensive hepatic O-demethylation to dextrorphan that is catalyzed by CYP2D6. This is the same enzyme that is responsible for polymorphic debrisoquine hydroxylation in humans (Schmid et al., Clin. Pharmacol. Ther., 1985; 38: 618-624). An alternate pathway is mediated primarily by CYP3A4 and N-demethylation to form 3-methoxymorphinan (Von Moltke et al., J. Pharm. Pharmacol., 1998; 50: 997-1004). Both DX and 3-methoxymorphinan can be further demethylated to 3-hydroxymorphinan that is then subject to glucuronidation. The metabolic pathway that converts DM to DX is dominant in the majority of the population and is the principle for using DM as a probe to phenotype individuals as CYP2D6 extensive and poor metabolizers (Kupfer et al., Lancet 1984; 2: 517-518; Guttendorf et al., Ther. Drug Monit., 1988; 10: 490-498). Approximately 7% of the Caucasian population shows the poor metabolizer phenotype, while the incidence of poor metabolizer phenotype in Chinese and Black African populations is lower (Droll et al., Pharmacogenetics, 1998; 8: 325-333). A study examining the ability of DM to increase pain threshold in extensive and poor metabolizers found antinociceptive effects of DM were significant in poor metabolizers but not in extensive metabolizers (Desmeules et al., J. Pharmacol. Exp. Ther., 1999; 288: 607-612). The results are consistent with direct effects of parent DM rather than the DX metabolite on neuromodulation.

It has long been known that in most people (estimated to include about 90% of the general population in the United States), dextromethorphan is rapidly metabolized and eliminated by the body (Ramachander et al., J. Pharm. Sci., 1977 July, 66(7):1047-8; and Vetticaden et al., Pharm. Res., 1989 Jan., 6(1):13-9). This elimination is largely due to an enzyme known as the P450 2D6 (or IID6) enzyme, which is one member of a class of oxidative enzymes that exist in high concentrations in the liver, known as cytochrome P450 enzymes (Kronbach et al., Anal. Biochem., 1987 April, 162(1):24-32; and Dayer et al., Clin. Pharmacol. Ther., 1989 Jan., 45(1):34-40). In addition to metabolizing dextromethorphan, the P450 2D6 isozyme also oxidizes sparteine and debrisoquine. It is known that the P450 2D6 enzyme can be inhibited by a number of drugs, particularly quinidine (Brinn et al., Br. J. Clin. Pharmacol., 1986 August, 22(2):194-7; Inaba et al., Br. J. Clin. Pharmacol., 1986 August, 22(2):199-200; Brosen et al., Pharmacol. Toxicol., 1987 April, 60(4):312-4; Otton et al., Drug Metab. Dispos., 1988 Jan-February, 16(1):15-7; Otton et al., J. Pharmacol. Exp. Ther., 1988 October, 247(1):242-7; Funck-Brentano et al., Br. J. Clin. Pharmacol., 1989 April, 27(4):435-44; Funck-Brentano et al., J. Pharmacol. Exp. Ther., 1989 April, 249(1):134-42; Nielsen et al., Br. J. Clin. Pharmacol., 1990 March, 29(3):299-304; Broly et al., Br. J. Clin. Pharmacol., 1989 July, 28(1):29-36).

Patients who lack the normal levels of P450 2D6 activity are classified in the medical literature as “poor metabolizers,” and doctors are generally warned to be cautious about administering various drugs to such patients. “The diminished oxidative biotransformation of these compounds in the poor metabolizer (PM) population can lead to excessive drug accumulation, increased peak drug levels, or in some cases, decreased generation of active metabolites . . . . Patients with the PM phenotype are at increased risk of potentially serious untoward effects . . . ” (Guttendorf et al., Ther. Drug Monit., 1988, 10(4):490-8, page 490). Accordingly, doctors are cautious about administering quinidine to patients, and rather than using drugs such as quinidine to inhibit the rapid elimination of dextromethorphan, researchers working in this field have administered very large quantities (such as 750 mg/day) of dextromethorphan to their patients, even though this is known to introduce various problems (Walker et al., Clin Neuropharmacol., 1989 August, 12(4):322-30; and Albers et al., Stroke, 1991 August, 22(8):1075-7).

A number of in vitro studies have been undertaken to determine the types of drugs that inhibit CYP2D6 activity. Quinidine (Q) is one of the most potent of those that have been studied (Inaba et al., Br. J. Clin. Pharmacol., 1986; 22:199-200). These observations led to the hypothesis that concomitant dosing with Q could increase the concentration of DM in plasma.

Therefore, one approach for increasing systemically available DM is to coadminister the CYP2D6 inhibitor, quinidine, to protect DM from metabolism (Zhang et al., Clin. Pharmacol. Ther. 1992; 51: 647-655). Quinidine administration can convert subjects with extensive metabolizer phenotype to poor metabolizer phenotype (Inaba et al., Br. J. Clin. Pharmacol., 1986; 22: 199-200). When this combination therapy was tried in amyotrophic lateral sclerosis patients it appeared to exert a palliative effect on symptoms of pseudobulbar affect (Smith et al., Neurol., 1995; 54: 604P). Combination treatment with DM and quinidine also appeared effective for patients with chronic pain that could not be adequately controlled with other medications. This observation is consistent with a report that showed DM was effective in increasing pain threshold in poor metabolizers and in extensive metabolizers given quinidine, but not in extensive metabolizers (Desmeules et al., J. Pharmacol. Exp. Ther., 1999; 288: 607-612). To date, most studies have used quinidine doses ranging from 50 to 200 mg to inhibit CYP2D6 mediated drug metabolism, but no studies have identified a minimal dose of quinidine for enzyme inhibition.

Rapid dextromethorphan elimination may be overcome by co-administration of quinidine along with dextromethorphan (U.S. Pat. No. 5,206,248 to Smith). The chemical structure of quinidine is as follows:

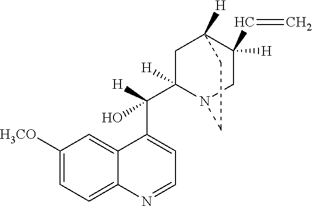

Quinidine co-administration has at least two distinct beneficial effects. First, it greatly increases the quantity of dextromethorphan circulating in the blood. In addition, it also yields more consistent and predictable dextromethorphan concentrations. Research involving dextromethorphan or co-administration of dextromethorphan and quinidine, and the effects of quinidine on blood plasma concentrations, are described in the patent literature (U.S. Pat. No. 5,166,207, U.S. Pat. No. 5,863,927, U.S. Pat. No. 5,366,980, U.S. Pat. No. 5,206,248, and U.S. Pat. No. 5,350,756 to Smith).

In addition, the results obtained to date suggest that dextromethorphan is likely to be useful for treating some cases of emotional lability which are due to administration of other drugs. For example, various steroids, such as prednisone, are widely used to treat autoimmune diseases such as lupus. However, prednisone has adverse events on the emotional state of many patients, ranging from mild but noticeably increased levels of moodiness and depression, up to severely aggravated levels of emotional lability that can impair the business, family, or personal affairs of the patient.

In addition, dextromethorphan in combination with quinidine can reduce the external displays or the internal feelings that are caused by or which accompany various other problems such as “premenstrual syndrome” (PMS), Tourette's syndrome, and the outburst displays that occur in people suffering from certain types of mental illness. Although such problems may not be clinically regarded as emotional lability, they involve manifestations that appear to be sufficiently similar to emotional lability to suggest that dextromethorphan can offer an effective treatment for at least some patients suffering from such problems.

One of the significant characteristics of the treatments of preferred embodiments is that the treatments function to reduce emotional lability without tranquilizing or otherwise significantly interfering with consciousness or alertness in the patient. As used herein, “significant interference” refers to adverse events that would be significant either on a clinical level (they would provoke a specific concern in a doctor or psychologist) or on a personal or social level (such as by causing drowsiness sufficiently severe that it would impair someone's ability to drive an automobile). In contrast, the types of very minor side effects that can be caused by an over-the-counter drug such as a dextromethorphan-containing cough syrup when used at recommended dosages are not regarded as significant interference.

The magnitude of a prophylactic or therapeutic dose of dextromethorphan in combination with quinidine in the acute or chronic management of emotional lability or other chronic conditions can vary with the particular cause of the condition, the severity of the condition, and the route of administration. The dose and/or the dose frequency can also vary according to the age, body weight, and response of the individual patient.

It can be preferred to administer dosages outside of these preferred ranges in some cases, as will be apparent to those skilled in the art. Further, it is noted that the ordinary skilled clinician or treating physician will know how and when to interrupt, adjust, or terminate therapy in consideration of individual patient response.

Any suitable route of administration can be employed for providing the patient with an effective dosage of dextromethorphan in combination with quinidine. For example, oral, rectal, transdermal, parenteral (subcutaneous, intramuscular, intravenous), intrathecal, topical, inhalable, and like forms of administration can be employed. Suitable dosage forms include tablets, troches, dispersions, suspensions, solutions, capsules, patches, and the like. Administration of medicaments prepared from the compounds described herein can be by any suitable method capable of introducing the compounds into the bloodstream. Formulations of preferred embodiments can contain a mixture of active compounds with pharmaceutically acceptable carriers or diluents as are known by those of skill in the art.

The present method of treatment of emotional lability can be enhanced by the use of dextromethorphan in combination with quinidine as an adjuvant to known therapeutic agents, such as fluoxetine hydrochloride, marketed as PROZAC® by Eli Lilly and Company, and the like. Preferred adjuvants include pharmaceutical compositions conventionally employed in the treatment of the disordered as discussed herein.

The pharmaceutical compositions of the present invention comprise dextromethorphan in combination with quinidine, or pharmaceutically acceptable salts of dextromethorphan and/or quinidine, as the active ingredient and can also contain a pharmaceutically acceptable carrier, and optionally, other therapeutic ingredients.

The terms “pharmaceutically acceptable salts” or “a pharmaceutically acceptable salt thereof” refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as The Merck Index. Any suitable constituent can be selected to make a salt of an active drug discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives of dextromethorphan and/or quinidine can also be suitable for use in compositions and methods of preferred embodiments. In particularly preferred embodiments, the dextromethorphan is administered in the form of dextromethorphan hydrobromide, and the quinidine is administered in the form of quinidine sulfate. For example, a dose of 30 mg dextromethorphan hydrobromide (of molecular formula C₁₈H₂₅NO.HBr.H₂O) and 30 quinidine sulfate (of molecular formula (C₂₀H₂₄N₂O₂)₂.H₂SO₄.2H₂O) may be administered (corresponding to an effective dosage of approximately 22 mg dextromethorphan and 25 mg quinidine). Other preferred dosages include, for example, 45 mg dextromethorphan hydrobromide and 30 quinidine sulfate (corresponding to an effective dosage of approximately 33 mg dextromethorphan and approximately 25 mg quinidine); 60 mg dextromethorphan hydrobromide and 30 quinidine sulfate (corresponding to an effective dosage of approximately 44 mg dextromethorphan and approximately 25 mg quinidine); 45 mg dextromethorphan hydrobromide and 45 quinidine sulfate (corresponding to an effective dosage of approximately 33 mg dextromethorphan and 37.5 mg quinidine); 60 mg dextromethorphan hydrobromide and 60 quinidine sulfate (corresponding to an effective dosage of approximately 44 mg dextromethorphan and 50 mg quinidine).

The compositions can be prepared in any desired form, for example, tables, powders, capsules, suspensions, solutions, elixirs, and aerosols. Carriers such as starches, sugars, microcrystalline cellulose, diluents, granulating agents, lubricants, binders, disintegrating agents, and the like can be used in oral solid preparations. Oral solid preparations (such as powders, capsules, and tablets) are generally preferred over oral liquid preparations. However, in certain embodiments oral liquid preparations can be preferred over oral solid preparations. The most preferred oral solid preparations are tablets. If desired, tablets can be coated by standard aqueous or nonaqueous techniques.

In addition to the common dosage forms set out above, the compounds can also be administered by sustained release, delayed release, or controlled release compositions and/or delivery devices, for example, such as those described in U.S. Pat. Nos. 3,845,770; 3,916,899; 3,536,809; 3,598,123; and 4,008,719.

Pharmaceutical compositions suitable for oral administration can be provided as discrete units such as capsules, cachets, tablets, and aerosol sprays, each containing predetermined amounts of the active ingredients, as powder or granules, or as a solution or a suspension in an aqueous liquid, a non-aqueous liquid, an oil-in-water emulsion, or a water-in-oil liquid emulsion. Such compositions can be prepared by any of the conventional methods of pharmacy, but the majority of the methods typically include the step of bringing into association the active ingredients with a carrier which constitutes one or more ingredients. In general, the compositions are prepared by uniformly and intimately admixing the active ingredients with liquid carriers, finely divided solid carriers, or both, and then, optionally, shaping the product into the desired presentation.

For example, a tablet can be prepared by compression or molding, optionally, with one or more additional ingredients. Compressed tablets can be prepared by compressing in a suitable machine the active ingredient in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet contains from about 30 mg to about 60 mg of dextromethorphan and from about 30 mg to about 45 mg quinidine, and each capsule contains from about 30 mg to about 60 mg of dextromethorphan and from about 30 mg to about 45 mg quinidine. Most preferably, tablets or capsules are provided in a range of dosages to permit divided dosages to be administered. For example, tablets, cachets or capsules can be provided that contain about 5 mg dextromethorphan and about 1, 2, 3, 4, 5, 7.5, 10, or 15 mg quinidine; about 10 mg dextromethorphan and about 1, 2, 3, 4, 5, 7.5, 10, or 15 mg quinidine; about 15 mg dextromethorphan and about 1, 2, 3, 4, 5, 7.5, 10, or 15 mg quinidine; about 20 mg dextromethorphan and about 1, 2, 3, 4, 5, 7.5, 10, 15, 20 or 30 mg quinidine; about 25 mg dextromethorphan and about 1, 2, 3, 4, 5, 7.5, 10, 15, 20 or 30 mg quinidine; about 30 mg dextromethorphan and about 5, 7.5, 10, 15, 30, or 45 mg quinidine; and the like. A dosage appropriate to the patient, the condition to be treated, and the number of doses to be administered daily can thus be conveniently selected. While it is generally preferred to incorporate both dextromethorphan and quinidine in a single tablet or other dosage form, in certain embodiments it can be desirable to provide the dextromethorphan and quinidine in separate dosage forms.

Patients suffering from emotional lability and other conditions as described herein can be treated with dextromethorphan in combination with an amount of quinidine substantially lower than the minimum amount heretofore believed to be necessary to provide a significant therapeutic effect. As used herein, a “minimum effective therapeutic amount” is that amount which provides a satisfactory degree of inhibition of the rapid elimination of dextromethorphan from the body, while producing no adverse effect or only adverse events of an acceptable degree and nature. More specifically, a preferred effective therapeutic amount is within the range of from about 20, 25 or 30 mg to about 60 mg of dextromethorphan and less than about 50 mg of quinidine per day, preferably about 20 or 30 mg to about 60 mg of dextromethorphan and about 30 mg to about 45 mg of quinidine per day, the amount being preferably administered in a divided dose based on the plasma half-life of dextromethorphan. For example, in a preferred embodiment dextromethorphan and quinidine are administered in specified mg increments to achieve a target concentration of dextromethorphan of a specified level in μg/mL plasma, with a maximum preferred specified dosage of dextromethorphan and quinidine based on body weight. The target dose is then preferably administered every 12 hours. Since the level of quinidine is minimized, the side effects observed at high dosages for quinidine are minimized or eliminated, a significant benefit over compositions containing dextromethorphan in combination with higher levels of quinidine.

The combination of dextromethorphan and quinidine of preferred embodiments can also be extremely effective in formulations for the treatment for other chronic disorders which do not respond well to other treatments. Dextromethorphan in combination with quinidine can be used to effectively treat severe or intractable coughing, which has not responded adequately to non-addictive, non-steroid medications, with minimal side-effects. Intractable coughing is a consequence of respiratory infections, asthma, emphysema, and other conditions affecting the pulmonary system.

Dextromethorphan in combination with quinidine as in the preferred embodiments can also be used in pharmaceutical compositions for treating dermatitis. As used herein, “dermatitis” or “eczema” is a skin condition characterized by visible skin lesions and/or an itching or burning sensation on the skin. Dextromethorphan in combination with quinidine as in the preferred embodiments can also be used in pharmaceutical compositions for the treatment of chronic pain from conditions such as stroke, trauma, cancer, and pain due to neuropathies such as herpes zoster infections and diabetes. Other conditions that can be treated using dextromethorphan in combination with quinidine according to the preferred embodiments can include sexual dysfunctions, such as priapism or premature ejaculation, as well as tinnitus.

Certain side effects are associated with the administration of dextromethorphan and/or quinidine. Side-effects of dextromethorphan use can include, but are not limited to, body rash/itching, nausea, drowsiness, dizziness, fever, vomiting, blurred vision, dilated pupils, sweating, hypertension, shallow respiration, diarrhea, and urinary retention. Side-effects of quinidine use can include, but are not limited to, abdominal pain, diarrhea, hepatitis, inflammation of the esophagus (gullet), loss of appetite, nausea, cinchonism, blurred or double vision, confusion, delirium, headache, intolerance to light, hearing loss, ringing in the ears, vertigo, and vomiting. Side-effects of a dextromethorphan and quinidine combination therapy can include, but are not limited to, anorexia, anxiety, arthralgia, constipation, confusion, diarrhea, dizziness (excluding vertigo), dyspnea, edema lower limb, fall, fatigue, flatulence, headache, hypertonia, joint stiffness, localized infection, loose stools, muscle cramps, muscle spasms, nasopharyngitis, nausea, pruritus, sinus congestion, sleep disorder, somnolence, sweating increased, upper respiratory tract infection, vomiting, and weakness.

One aspect of the invention therefore relates to methods of improving the safety and tolerability of a dextromethorphan and quinidine combination therapy. In some embodiments, the method of the present invention reduces side effects associated with a long term, dextromethorphan/low-dose quinidine combination therapy. In some embodiments, the method of the present invention reduces CNS side effects. In some embodiments, the method of the present invention reduces gastrointestinal side effects. In some embodiments, the method of the present invention reduces CNS and gastrointestinal side effects.

The methods of the present invention can be used to reduce nausea associated with a long term, dextromethorphan/low-dose quinidine combination therapy. In some embodiments, the methods of the present invention can be used to reduce dizziness associated with a long term, dextromethorphan/low-dose quinidine combination therapy. In some embodiments, the methods of the present invention can be used to reduce fatigue associated with a long term, dextromethorphan/low-dose quinidine combination therapy. In some embodiments, the methods of the present invention can be used to reduce side effects associated with a long term, dextromethorphan/low-dose quinidine combination therapy, such as anorexia, anxiety, arthralgia, constipation, confusion, diarrhea, dizziness (excluding vertigo), dyspnea, edema lower limb, fall, fatigue, flatulence, headache, hypertonia, joint stiffness, localized infection, loose stools, muscle cramps, muscle spasms, nasopharyngitis, nausea, pruritus, sinus congestion, sleep disorder, somnolence, sweating increased, upper respiratory tract infection, vomiting, weakness, urinary tract infection, muscle weakness, dysphagia, and pain in extremity.

In some embodiments, the method of reducing CNS and gastrointestinal side effects associated with a long term, dextromethorphan/low-dose quinidine combination therapy comprising permitting a patient to acclimate to dextromethorphan said method comprising administration of a sub-optimal combination dose of for a period of no less than 7 days and no more than 20 days prior to increasing the dose of dextromethorphan to a therapeutically beneficial amount, where the sub-optimal combination dose comprises dextromethorphan from about 10 mg/day to about 30 mg/day and quinidine from about 5 mg/day to about 15 mg/day with the proviso that the weight to weight ratio of dextromethorphan to quinidine is 1:0.75 or less (e.g., 1:0.5, 1:0.4, 1:0.3) of quinidine. In other words, for each weight unit of dextromethorphan, there should be no more than ¾ unit of quinidine. In some embodiments, the weight to weight ratio of dextromethorphan to quinidine is 1:0.5 or less of quinidine, i.e., for each weight unit of dextromethorphan, there should be no more than ½ A unit of quinidine.

As defined herein the term “therapeutically beneficial amount” or “therapeutically beneficial dose” refers to a dose that produces the effects for which it is administered. The exact dose will depend on the purpose of the treatment, e.g., treating emotional lability or pseudobulbar effect, treating chronic or intractable pain, treating tinnitus, treating sexual dysfunction, treating intractable coughing, and treating dermatitis. Common dose determining techniques are disclosed, e.g., in Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins.

In some embodiments, a therapeutically beneficial amount or dose refers to the amount sufficient to treat emotional lability or pseudobulbar effect. In some embodiment, a therapeutically beneficial amount or dose comprises dextromethorphan from about 20 mg/day to about 60 mg/day and quinidine from about 10 mg/day to about 30 mg/day with the proviso that the weight to weight ratio of dextromethorphan to quinidine is 1:0.75 or less of quinidine. In other words, for each weight unit of dextromethorphan, there should be no more than ¾ unit of quinidine. In some embodiments, the weight to weight ratio of dextromethorphan to quinidine is 1:0.5 or less of quinidine, i.e., for each weight unit of dextromethorphan, there should be no more than ½ unit of quinidine. In some embodiments, the pseudobulbar affect or emotional lability is caused by a neurodegenerative disease or condition or a brain injury. The therapeutically beneficial amount or dose can be administered as one combined dose per day, or as at least two combined doses per day. The dextromethorphan and quinidine can also be administered in separate doses. In some embodiments, the amount of quinidine administered comprises from about 20 mg/day to about 30 mg/day. In some embodiments, the amount of dextromethorphan administered comprises from about 20 mg/day to about 60 mg/day.

In some embodiments, a therapeutically beneficial amount or dose refers to the amount sufficient to treat chronic or intractable pain. In some embodiments, a therapeutically beneficial amount or dose refers to the amount sufficient to treat tinnitus. In some embodiments, a therapeutically beneficial amount or dose refers to the amount sufficient to treat sexual dysfunction. In some embodiments, a therapeutically beneficial amount or dose refers to the amount sufficient to treat intractable coughing. In some embodiments, a therapeutically beneficial amount or dose refers to the amount sufficient to treat dermatitis.

The sub-optimal combination dose can be a fraction of the therapeutically beneficial amount or dose, e.g., 1-25%, 1-33%, 1-50%, 1-67%, or 1-75%. In some embodiments, the sub-optimal combination dose is 10%, 25%, 33%, 50%, 67%, or 75% of the therapeutically beneficial dose. In some cases, the amount of dextromethorphan in the sub-optimal combination dose is 10%, 25%, 33%, 50%, 67%, or 75% of the amount dextromethorphan in the therapeutically beneficial dose.

In some embodiments, the sub-optimal combination dose according to the method of the present invention is administered as one combined dose per day. In some embodiments, the sub-optimal combination dose is administered as at least two combined dose per day. the sub-optimal combination dose is administered as at least three combined dose per day. In some embodiments, the sub-optimal combination dose comprises the dextromethorphan and the quinidine administered in separate doses.

In some embodiments, the weight to weight ratio of dextromethorphan to quinidine in the sub-optimal combination dose is 1:0.75 or less of quinidine. In some embodiments, the weight to weight ratio of dextromethorphan to quinidine in the sub-optimal combination dose is 1:0.5 of quinidine. In some embodiments, the weight to weight ratio of dextromethorphan to quinidine in the sub-optimal combination dose is less than 1:0.5 of quinidine. In some embodiments, the weight to weight ratio of dextromethorphan to quinidine in the sub-optimal combination dose is 1:0.45 or less of quinidine, 1:0.4 or less of quinidine, 1:0.35 or less of quinidine, 1:0.3 or less of quinidine, 1:0.25 or less of quinidine, 1:0.2 or less of quinidine, 1:0.15 or less of quinidine, 1:0.1 or less of quinidine, 1:0.05 or less of quinidine.

In some embodiments, the sub-optimal combination dose is administered for a period of 7 days. In some embodiments, the sub-optimal combination dose is administered for a period of 14 days. In some embodiments, the sub-optimal combination dose is administered for a period of 8, 9, 10, 11, 12, 13, 15, 16, 17, 18, 19, or 20 days.

In some embodiments, the sub-optimal combination dose comprises dextromethorphan from about 10 mg/day to about 30 mg/day. In some embodiments, the sub-optimal combination dose comprises dextromethorphan from about 10 mg/day to about 20 mg/day. In some embodiments, the sub-optimal combination dose comprises dextromethorphan from about 20 mg/day to about 30 mg/day. In some embodiments, the sub-optimal combination dose comprises dextromethorphan about 10 to about 15 mg/day, about 15 to about 20 mg/day, about 20 to about 25 mg/day, about 25 to about 30 mg/day. In some embodiments, the sub-optimal combination dose comprises dextromethorphan about 10 mg/day, about 15 mg/day, about 20 mg/day, about 25 mg/day, about 30 mg/day.

In some embodiments, the sub-optimal combination dose comprises quinidine from about 5 mg/day to about 15 mg/day. In some embodiments, the sub-optimal combination dose comprises quinidine from about 5 mg/day to about 10 mg/day. In some embodiments, the sub-optimal combination dose comprises quinidine from about 10 mg/day to about 15 mg/day. In some embodiments, the sub-optimal combination dose comprises quinidine about 5 to about 7.5 mg/day, about 7.5 to about 10 mg/day, about 10 to about 12.5 mg/day, about 12.5 to about 15 mg/day. In some embodiments, the sub-optimal combination dose comprises quinidine about 5 mg/day, about 7.5 mg/day, about 10 mg/day, about 12.5 mg/day, about 15 mg/day.

In some embodiments, the sub-optimal combination dose comprises dextromethorphan about 30 mg/day and quinidine about 10 mg/day. In some embodiments, the sub-optimal combination dose comprises dextromethorphan about 20 mg/day and quinidine about 10 mg/day. In some embodiments, the sub-optimal combination dose comprises dextromethorphan about 30 mg/day and quinidine about 5 mg/day. In some embodiments, the sub-optimal combination dose comprises dextromethorphan about 30 mg/day and quinidine about 15 mg/day. In some embodiments, the sub-optimal combination dose comprises dextromethorphan about 20 mg/day and quinidine about 5 mg/day. In some embodiments, the sub-optimal combination dose comprises dextromethorphan about 10 mg/day and quinidine about 5 mg/day.

In some embodiments, the method of the present invention reduces side effects associated with a long term, dextromethorphan/low-dose quinidine combination therapy for treatment of emotional lability or pseudobulbar effect. In some embodiments, the emotional lability or pseudobulbar effect is caused by a neurodegenerative disease or condition or a brain injury. In some embodiments, the method of the present invention reduces side effects associated with a long term, dextromethorphan/low-dose quinidine combination therapy for treatment of chronic or intractable pain. In some embodiments, the method of the present invention reduces side effects associated with a long term, dextromethorphan/low-dose quinidine combination therapy for treatment of chronic pain resulting from stroke, cancer, or trauma as well as neuropathic pain. In some embodiments, the method of the present invention reduces side effects associated with a long term, dextromethorphan/low-dose quinidine combination therapy for treatment of tinnitus. In some embodiments, the method of the present invention reduces side effects associated with a long term, dextromethorphan/low-dose quinidine combination therapy for treatment of sexual dysfunction. In some embodiments, the method of the present invention reduces side effects associated with a long term, dextromethorphan/low-dose quinidine combination therapy for treatment of intractable coughing. In some embodiments, the method of the present invention reduces side effects associated with a long term, dextromethorphan/low-dose quinidine combination therapy for treatment of dermatitis.

Further provided herein are kits for reducing Central Nervous System (CNS) and gastrointestinal side effects associated with a long term, dextromethorphan/low-dose quinidine combination therapy. The kits can have one package with one dose, for example, a sub-optimal combination dose described herein. The kits can also have more than one doses, e.g., two doses. For example, the kits can have a sub-optimal combination dose described herein, and a therapeutically beneficial dose described herein, for example, 30 mg/day dextromethorphan in combination with 10 mg/day quinidine, or 20 mg/day dextromethorphan in combination with 10 mg/day quinidine. The sub-optimal combination dose can be a fraction of the therapeutically beneficial dose, e.g., 1-25%, 1-33%, 1-50%, 1-67%, or 1-75%. In some embodiments, the sub-optimal combination dose is 10%, 25%, 33%, 50%, 67%, or 75% of the therapeutically beneficial dose. In some cases, the amount of dextromethorphan in the sub-optimal combination dose is 10%, 25%, 33%, 50%, 67%, or 75% of the amount dextromethorphan in the therapeutically beneficial dose.

### EXAMPLES

### Example 1

### A Double-Blind, Randomized, Placebo-Controlled, Multicenter Study to Assess the Safety and Efficacy and to Determine the Pharmacokinetics of Two Doses of AVP-923 (Dextromethorphan/Quinidine) in the Treatment of Pseudobulbar Affect (PBA) in Patients with Amyotrophic Lateral Sclerosis and Multiple Sclerosis

This was a multicenter, randomized, double-blind, three-arm parallel, placebo-controlled study of 12 weeks duration comparing two different doses of AVP-923 to placebo followed by an optional open label treatment phase. The objectives of the study were to evaluate the safety, tolerability, and efficacy of two different doses of AVP-923 (capsules containing either 30 mg of dextromethorphan hydrobromide and 10 mg of quinidine sulfate [AVP-923-30] or 20 mg of dextromethorphan hydrobromide and 10 mg of quinidine sulfate [AVP-923-20]) when compared to placebo, for the treatment of PBA in a population of patients with amyotrophic lateral sclerosis (ALS) or multiple sclerosis (MS) over a 12-week period. An additional objective was to determine the pharmacokinetic parameters of the two different doses of AVP-923 in a subset of the study population.

Male and female patients, between 18 and 80 of age, with clinically diagnosed Pseudobulbar Affect (PBA) as a result of an underlying neurological disorder (amyotrophic lateral sclerosis [ALS] or multiple sclerosis [MS]) were screened and selected. Patients must score 13 or higher on the Center for Neurologic Studies-Lability Scale (CNS-LS) to be eligible to participate in the study. An estimated number of 326 patients (approximately 197 patients with ALS and 129 patients with MS were enrolled at approximately 60 sites (40 US sites and 20 international sites).

Eligible patients were randomized in a double-blind manner to receive treatment with either one of the two different doses of AVP-923 (AVP-923-30/10 [DMQ 30/10] or AVP-923-20/10 [DMQ 20/10]) or placebo. Beginning with a morning dose on Day 1, patients received either a determined dose of AVP-923 or identical-appearing placebo capsules, and continued to take one capsule in the morning during the first 7 days (week 1) of the study, and then the patients started taking one capsule two times a day (every 12 hours) for the remaining 11 weeks of the study, to complete a total of 84 days (12 weeks). The last day (Day 84) was the last day the patient was on study and may occur anywhere between Day 81 a.m. and Day 84 a.m. Each patient were required to complete a diary recording the daily number of laughing and/or crying episodes experienced, medication schedule, and any adverse experiences throughout the entire study. (see Study Schedule of Events). Patients completing Day 84 of the study as scheduled were eligible to continue in a 12-week open label treatment phase where all patients will receive (DMQ 30/10) twice daily.

The following table summarizes the study treatment groups:

Treatment A (AVP-923-30): 30 mg of dextromethorphan hydrobromide USP and 10 mg of quinidine sulfate USP.

Treatment B: (AVP-923-20):20 mg of dextromethorphan hydrobromide USP and 10 mg of quinidine sulfate USP)

Treatment C: Placebo

Based on PBA episode rates in previous studies of DM/Q for PBA in ALS (Brooks, et al., Neurology, 63:1364-1370, 2004) and in MS (Panitch et al., Ann Neurol, 59:780-787, 2006) a sample size of approximately 90 patients (60 with ALS and 30 with MS) per treatment group was planned. This size was expected to be sufficient to detect a 36% reduction in mean episode rate for DMQ 30/10 vs. placebo with at least 90% power. The study was not powered to test a difference between DMQ 30/10 and DMQ 20/10.

The primary efficacy endpoint was the number of laughing and/or crying episodes as recorded in the patient diary. The primary efficacy analysis was based on the changes in laughing/crying episode rates recorded in the patient diary estimated using negative binomial regression on the daily episode counts. Episode counts were reported and analyzed as a rate expressed as episodes per day.

The secondary endpoints included: 1) mean change in CNS-LS score, 2) mean change in Neuropsychiatric Inventory Questionnaire (NPI-Q), 3) mean change in SF-36 Health Survey (SF-36), 4) mean change in the Beck Depression Inventory (BDI-II), and 5) mean change in Pain Rating Scale score (MS patients only). Secondary endpoints were analyzed using analysis of covariance based on changes from baseline, adjusting for study site and baseline levels.

Additional analyses to clarify clinical understanding of the treatments and/or generalizability of the findings were performed and included: (1) Time to onset of action; (2) Number of episode-free days; (3) Percentage of patients showing remission (no episodes during the last 14 days of study participation); (4) Percentage of patients showing a clinical response (defined as a 40% decrease in number of episodes at the end of the study).

As an additional evaluation of the study, caregiver burden was also assessed. The Caregiver Strain Index (CSI) was administered to the patient caregiver at Baseline visit and at the end of the study. CSI is a self-administered 13-question tool that measures strain related to care provision.

Safety and tolerance of AVP-923 was determined by reporting adverse events; physical examinations, vital signs (including blood pressure, heart rate, respiratory rate and body temperature), resting diurnal oxygen saturation, nocturnal oxygen saturation assessments, 12-lead ECGs with a 2-minute rhythm strip, and clinical assessment of clinical laboratory variables. Safety analyses were randomized by treatment.

All patients enrolled in the study underwent a blood draw at Visit 3 (Day 29) and at Visit 4 (Day 57) for analysis of plasma levels of dextromethorphan (DM), dextrorphan (DX) and quinidine (Q). A sub-set of 24 patients (approximately 16 patients with ALS and 8 patients with MS) in each treatment group were randomly assigned at specific sites to determine the pharmacokinetic profile of the investigational product. Blood samples were collected at Visit 3 (Day 29) at pre-specified time points over a 12-hour period. A blood specimen was collected at the Baseline Visit for CYP2D6 genotyping.

Patients who completed the Day 84 visit of protocol 07-AVR-123 according to protocol were allowed to enter an open-label extension of the study, in which they will receive AVP-923-30/10 (DMQ 30/10) twice-a-day (every twelve hours) for a 12-week period. The primary objective of the open-label extension phase was to assess the long-term safety of AVP-923 in patients diagnosed with PBA as a result of an underlying neurological disorder (ALS and MS). Patients were given a diary card to record any AEs and times the medication was taken each day. Efficacy assessments were completed at the clinic. Safety was assessed by physical examinations, vital signs, 12-lead ECGs with a 2-minute rhythm strip, and clinical laboratory tests.

The Baseline Visit occurred within 14 days of the last visit (Visit 5-Day 84) of the placebo controlled phase of Study 07-AVR-123. The following procedures performed during Visit 5 of the double-blind phase of Study 07-AVR-123 do not need to be repeated during the Baseline Visit of the open-label safety extension. These procedures are: clinical laboratory tests and physical exam.

Patients returned to the study site for study procedures and disease evaluation at Day 15 after entering into the open-label safety phase of the study, and then at Day 42 and Day 84 of the study for a total of 4 visits, including the Baseline Visit.

Both DMQ 30/10 and DMQ 20/10 provided a statistically significant reduction in episode rates over the course of the study when compared to placebo (p<0.0001). In an additional analysis of the primary endpoint, at week twelve (end of study), patients in the DMQ 30/10 group reported a statistically significant mean reduction of 88% from baseline in PBA episode rates (p=0.01).

The primary efficacy analysis was based on the changes from baseline in crying/laughing episode rates recorded in the patient diary. Episode counts were reported and analyzed as a rate expressed as episodes per day. The primary outcome was the additional reduction in episode rates experienced with DMQ 30/10 or DMQ 20/10 compared to placebo. In 326 randomized patients (of whom 283, or 86.8%, completed the study), the PBA-episode daily rate was 46.9% (p<0.0001) lower for DMQ 30/10 than for placebo and 49.0% (p<0.0001) lower for DMQ 20/10 than for placebo by longitudinal negative binomial regression, the pre-specified primary analysis. Mean CNS-LS scores decreased by 8.2 points for DMQ 30/10 and 8.2 for DMQ 20/10, compared to 5.7 for placebo (p=0.0002 and p=0.0113). Other endpoints showing statistically significant DMQ benefit included, for both dosage levels, the likelihood of PBA remission during the final 14 days and, for the higher dosage, improvement on measures of social functioning and mental health. Both dosages were safe and well tolerated.

An important secondary endpoint analysis was based on the change from baseline to end of study using the Center for Neurologic Studies Lability Scale (CNS-LS). The CNS-LS is a validated instrument measuring the frequency and severity of PBA. In this secondary endpoint analysis, patients receiving DMQ 30/10 reported a significantly greater reduction in mean CNS-LS score compared to patients who received placebo (p=0.0002).

Additional secondary endpoints included: 1) SF-36 Health Survey, 2) Neuropsychiatric Inventory Questionnaire (NPI-Q), 3) Beck Depression Inventory (BDI-II), and 4) Pain Rating Scale score (MS patients only).

Overall, both DMQ 30/10 and DMQ 20/10 were generally safe and well tolerated in the study. In the trial, 91.8%, 82.2% and 86.2% of patients completed the 12-week double blind phase of the study in the DMQ 30/10, DMQ 20/10, and placebo groups, respectively. The most common reason for early withdrawals was due to adverse events (AEs). Early withdrawal due to AEs occurred in 3.6%, 7.5% and 1.8% for the DMQ 30/10, DMQ 20/10, and placebo groups, respectively. The proportion of patients reporting at least one AE was 82.7% in the DMQ 30/10 group, 79.4% in the DMQ 20/10 group and 82.6% in the placebo group. Reported AEs were generally mild to moderate in nature. The most commonly reported AEs (>5% of patients) in the treatments groups are summarized in Table 1.

TABLE 1
Most Common Adverse Events (>5% of patients)*

| | DMQ 30/10 | DMQ 20/10 | Placebo |
|---|---|---|---|
| | N = 110 | N = 107 | N = 109 |
| Falls | 22 (20.0%) | 14 (13.1%) | 22 (20.2%) |
| Dizziness | 20 (18.2%) | 11 (10.3%) | 6 (5.5%) |
| Headache | 15 (13.6%) | 15 (14.0%) | 17 (15.6%) |
| Nausea | 14 (12.7%) | 8 (7.5%) | 10 (9.2%) |
| Diarrhea | 11 (10.0%) | 14 (13.1%) | 7 (6.4%) |
| Somnolence | 11 (10.0%) | 9 (8.4%) | 10 (9.2%) |
| Fatigue | 9 (8.2%) | 11 (10.3%) | 10 (9.2%) |
| Nasopharyngitis | 9 (8.2%) | 6 (5.6%) | 8 (7.3%) |
| Urinary tract infection | 8 (7.3%) | 4 (3.7%) | 3 (2.8%) |
| Constipation | 7 (6.4%) | 7 (6.5%) | 9 (8.3%) |
| Muscle Spasms | 7 (6.4%) | 4 (3.7%) | 10 (9.2%) |
| Muscle weakness | 6 (5.5%) | 5 (4.7%) | 4 (3.7%) |
| Dysphagia | 5 (4.5%) | 6 (5.6%) | 4 (3.7%) |
| Pain in extremity | 5 (4.5%) | 2 (1.9%) | 8 (7.3%) |
| Depression | 0 | 1 (0.9%) | 6 (5.5%) |

*The numbers in parenthesis represent percentage of patients in each treatment group reporting AEs.

The proportion of patients reporting at least one serious adverse event (SAE) was 7.3% in the DMQ 30/10 group, 8.4% in the DMQ 20/10 group and 9.2% in the placebo group. A total of 38 SAEs occurred in 27 patients over the course of the study. Of the 38 SAEs reported in the study, only two were deemed by the investigators to be possibly or probably treatment-related; zero in the DMQ 30/10 group, two in the DMQ 20/10 group and zero in the placebo group. In addition, there was a numerical difference in respiratory SAEs with five patients (4.7%) in the DMQ 30/10 group, three patients (2.9%) in the DMQ 20/10 group and two patients (1.9%) in the placebo group experiencing respiratory SAEs.

During the course of the study, no new cardiovascular safety signals were observed (Table 2). There were no clinically meaningful changes in QT interval, no reported pro-arrhythmic events and no reports of any cardiovascular SAEs.

TABLE 2
Electrophysiological Measures*

| | DMQ 30/10 | DMQ 20/10 | Placebo |
|---|---|---|---|
| Analysis of Central Tendency | N = 110 | N = 107 | N = 109 |
| Mean QTc - Baseline | 418.2/406.6 | 416.4/404.2 | 416.1/404.7 |
| (QTcB/QTcF) | | | |
| Mean QTc - Day 84 | 420.6/411.8 | 413.8/405.1 | 416.8/405.8 |
| (QTcB/QTcF) | | | |
| Mean A in Baseline to | 3.0/4.8 | −1.9/1.0 | 1.6/1.0 |
| Day 84 (QTcB/QTcF) | | | |

Outlier Categorical Analysis (Visit 2 through Visit 5)**

| | | | |
|---|---|---|---|
| Absolute > 450 msec | 6.3%/1.9% | 4.9%/1.2% | 6.1%/2.4% |
| (QTcB/QTcF) | | | |
| Absolute > 480 msec | 0.2%/0.0% | 0.0%/0.0% | 0.9%/0.0% |
| (QTcB/QTcF) | | | |
| Absolute > 500 msec | 0.0%/0.0% | 0.0%/0.0% | 0.2%/0.0% |
| (QTcB/QTcF) | | | |
| Δ 30-60 msec | 7.0%/7.2% | 3.9%/2.9% | 6.6%/3.5% |
| (QTcB/QTcF) | | | |
| Δ > 60 msec | 0.5%/0.0% | 0.2%/0.0% | 0.5%/0.5% |
| (QTcB/QTcF) | | | |
| Δ > 90 msec | 0.0%/0.0% | 0.0%/0.0% | 0.0%/0.0% |
| (QTcB/QTcF) | | | |

*QTc is the QT interval corrected for heart rate. QTcB is the QT interval with corrected heart rate using Bazzet formula, and QTcF is the QT interval with corrected heart rate using Fridericia formula.
**Percent of EKGs taken over the course of the study

### Example 2

### Reduced Side Effects Associated with Dextromethorphan/Quinidine Combination Therapy by Administering a Daily Dose of 30 Mg Dextromethorphan, 10 Mg Quinidine

Combination therapy of DMQ 30/10 and DMQ 20/10 with the administration of a sub-optimal combination dose (titration) for a period of time offers improved efficacy and decreased risk (Table 3). In this clinical study, patients were to take one capsule of AVP-923-30/10 (30 mg dextromethorphan, 10 mg quinidine), or one capsule of AVP-923-20/10 (20 mg dextromethorphan, 10 mg quinidine), or placebo for period of one week. Patients then started taking one capsule two times a day (every 12 hours) for the remaining 11 weeks. This titration dosing regimen provides a significant reduction in the occurrence of adverse effects, as compared to dosing regimens without titration dosing, e.g., a combination dose of 30 mg dextromethorphan, 30 mg quinidine, two times a day, every 12 hours (DMQ 30/30).

The proportion of patients reporting nausea was 13.1% in the DMQ 30/10 titration dosing regimen, and 79%-95% in the DMQ 30/30 regimen. The proportion of patients reporting dizziness was 18.7% in the DMQ 30/10 titration dosing regimen and 20%-26% in the DMQ 30/30 regimen. The proportion of patients reporting fatigue was 8.4% in the DMQ 30/10 titration dosing regimen and 14%-19% in the DMQ 30/30 regimen. Withdrawal due to AEs occurred in 3.7% of patients in the DMQ 30/10 titration dosing regimen, and 14%-24% of patients in the DMQ 30/30 regimen.

TABLE 3

| | DMQ 30/10 with | DMQ 30/30 without |
|---|---|---|
| | titration | titration* |
| Reduction in PBA | 88% decrease from | 79% (95%) decrease from |
| episodes | baseline | baseline |
| Percentage of patients | 12.7% | 22% (23%) |
| reporting nausea | | |
| Percentage of patients | 18.2% | 20% (26%) |
| reporting dizziness | | |
| Percentage of patients | 8.2% | 14% (19%) |
| reporting fatigue | | |
| All-cause | 8.2% | 26% (28%) |
| discontinuations | | |
| Discontinuations due | 3.6% | 14% (24%) |
| to side effects | | |

*The combination therapy using 30 mg dextromethorphan, 30 mg quinidine was carried out in two clinical studies. The numbers in parenthesis represent the results from a second clinical study.

The preferred embodiments have been described in connection with specific embodiments thereof. It will be understood that it is capable of further modification, and this application is intended to cover any variations, uses, or adaptations of the invention following, in general, the principles of the invention and including such departures from the present disclosure as come within known or customary practices in the art to which the invention pertains and as may be applied to the essential features hereinbefore set forth, and as fall within the scope of the invention and any equivalents thereof. All references cited herein, including but not limited to technical literature references and patents, are hereby incorporated herein by reference in their entireties.

What is claimed is:

1. A method of reducing Central Nervous System (CNS) and gastrointestinal side effects associated with a long term, dextromethorphan/low-dose quinidine combination therapy by permitting a patient to acclimate to dextromethorphan, the method comprising administrating to the patient of a sub-optimal combination dose for a period of no less than 7 days and no more than 20 days prior to increasing the dose of dextromethorphan to a therapeutically beneficial amount, wherein the sub-optimal combination dose comprises dextromethorphan from about 10 mg/day to about 30 mg/day and quinidine from about 5 mg/day to about 15 mg/day with the proviso that the weight to weight ratio of dextromethorphan to quinidine is 1:0.75 or less of quinidine.

2. The method of claim 1, wherein the weight to weight ratio of dextromethorphan to quinidine is 1:0.5 or less of quinidine.

3. The method of claim 1, wherein the side effect is nausea.

4. The method of claim 1, wherein the side effect is dizziness.

5. The method of claim 1, wherein the side effect is fatigue.

6. The method of claim 1, wherein the sub-optimal combination dose is administered as one combined dose per day.

7. The method of claim 1, wherein the sub-optimal combination dose is administered as at least two combined dose per day.

8. The method of claim 1, wherein the sub-optimal combination dose comprises the dextromethorphan and the quinidine administered in separate doses.

9. The method of claim 1, wherein the sub-optimal combination dose is administered for a period of 7 days.

10. The method of claim 1, wherein the sub-optimal combination dose is administered for a period of 14 days.

11. The method of claim 1, wherein the sub-optimal combination dose comprises from about 10 mg/day to about 20 mg/day dextromethorphan.

12. The method of claim 1, wherein the sub-optimal combination dose comprises from about 20 mg/day to about 30 mg/day dextromethorphan.

13. The method of claim 1, wherein the sub-optimal combination dose comprises about 10 mg/day dextromethorphan.

14. The method of claim 1, wherein the sub-optimal combination dose comprises about 20 mg/day dextromethorphan.

15. The method of claim 1, wherein the sub-optimal combination dose comprises about 30 mg/day dextromethorphan.

16. The method of claim 1, wherein the sub-optimal combination dose comprises from about 5 mg/day to about 10 mg/day quinidine.

17. The method of claim 1, wherein the sub-optimal combination dose comprises from about 10 mg/day to about 15 mg/day quinidine.

18. The method of claim 1, wherein the sub-optimal combination dose comprises about 5 mg/day quinidine.

19. The method of claim 1, wherein the sub-optimal combination dose comprises about 10 mg/day quinidine.

20. The method of claim 1, wherein the sub-optimal combination dose comprises about 15 mg/day quinidine.

21. The method of claim 1, wherein the sub-optimal combination dose comprises about 30 mg/day dextromethorphan and about 10 mg/day quinidine.

22. The method of claim 1, wherein the sub-optimal combination dose comprises about 20 mg/day dextromethorphan and about 10 mg/day quinidine.

23. The method of claim 1, wherein the dextromethorphan/quinidine combination therapy is for treatment of emotional lability or pseudobulbar effect.

24. The method of claim 23, wherein the emotional lability or pseudobulbar effect is caused by a neurodegenerative disease or condition or a brain injury.

25. The method of claim 1, wherein the sub-optimal combination dose is 50% of the therapeutically beneficial amount.

26. The method of claim 1, wherein the sub-optimal combination dose is one third of the therapeutically beneficial amount.

27. A kit for reducing Central Nervous System (CNS) and gastrointestinal side effects associated with a long term, dextromethorphan/low-dose quinidine combination therapy, comprising:
(a) a sub-optimal combination dose for a period of no less than 7 days and no more than 20 days comprising dextromethorphan from about 10 mg/day to about 30 mg/day and quinidine from about 5 mg/day to about 15 mg/day with the proviso that the weight to weight ratio of dextromethorphan to quinidine is 1:0.75 or less of quinidine; and
(b) a therapeutically beneficial dose for a period of 7 days or more.

28. The kit of claim 27, wherein the weight to weight ratio of dextromethorphan to quinidine is 1:0.5 or less of quinidine.

29. The kit of claim 27, wherein the sub-optimal combination dose is 50% of the therapeutically beneficial dose.

30. The kit of claim 27, wherein the sub-optimal combination dose is one third of the therapeutically beneficial dose.
